(19) United States
(12) Patent Application Publication
(10) Pub. No.: US 2011/0028867 A1
(43) Pub. Date: Feb. 3, 2011

(54) APPARATUS AND METHOD FOR NON-INVASIVE DELIVERY AND TRACKING OF FOCUSED ULTRASOUND GENERATED FROM TRANSDUCER

(75) Inventors: Seh-Eun Choo, Seoul (KR); Jae Chun Yang, Seoul (KR)

Correspondence Address: EDWARDS ANGELL PALMER & DODGE LLP, P.O. BOX 55874, BOSTON, MA, 02205, US

(73) Assignee: Seh-Eun Choo, Seoul (KR)

(21) Appl. No.: 12/845,210

(22) Filed: Jul. 28, 2010

(30) Foreign Application Priority Data
Jul. 29, 2009 (KR) 10-2009-0069385

(51) Int. Cl. A61N 7/00

(52) U.S. Cl. 601/2

(57) ABSTRACT

The present invention provides an apparatus for non-invasive delivery of focused ultrasound to a targeted area of a biological tissue, and a method thereof. The apparatus comprises an ultrasound wave generator to provide an amplified modulated waveform, a resonance circuit for tuning the waveform to have a predetermined frequency, an ultrasound transducer for generating a focused beam of the tuned waveform, an applicator supporting the ultrasound transducer, and an ultrasound-tissue coupling bag detachably mounted to the ultrasound transducer.

### CROSS-REFERENCE TO RELATED APPLICATION

This application claims under 35 U.S.C. §119(a) the benefit of Korean Application No. 10-2009-0069385 filed Jul. 29, 2009, the entire contents of which are incorporated herein by reference.

### BACKGROUND

(a) Technical Field

The present disclosure relates to an apparatus and a method of non-invasive delivery and tracking of focused ultrasound (FUS). More particularly, it relates to an apparatus and a method for delivering low-intensity FUS generated by an ultrasound transducer to a targeted position and tracking the focus of the ultrasound transducer in space for modification of an electrically-excitable tissue including neural tissues of an animal or human brain.

(b) Background Art

A human brain largely consists of cortical gray matter, subcortical brain structures, as well as the white matter tracts that link and relay the neuronal information between these brain areas. Through the systematic activation and deactivation of these brain areas, orchestrated with the modulation of the function of the neurotransmitters between neuronal cells/tissues, perception/cognition is achieved along with their physical manifestation.

There are several ways to modify the neuronal tissues function, either central or peripheral. One of the methods is to administer a chemical compound in the form of a pharmaceutical agent to reduce or enhance the excitability of the neural tissues or the degree of effects of the function of the neurotransmitters. However, the administration of the chemical compound cannot modulate a specific area of the brain since excitabilities of the neural tissues are different depending on their types and also the neurotransmitters are distributed across the brain.

In order to modulate a specific area, electronic electrodes are implanted directly to the area of interest through invasive surgery. For example, electrocorticogram (eCog) or deep brain stimulation (DBS) utilizes surgical implantation of an array of microelectrodes and concurrent stimulation of a local neural tissue to induce reversible or permanent modification of its function.

Another method to modulate a specific area is transcranial magnetic stimulation (TMS) technique. TMS utilizes the application of rapidly-switching strong magnetic field over the scalp and to induce the electrical current under the scalp and concurrently change the function of the neural tissue. However, not only the stimulated area under the scalp is too large (e.g., 2-3 cm in diameter) but also the stimulated depth is shallow from the scalp surface (e.g., 1-2 centimeters) due to steep reduction in the strength of the magnetic field from the surface of the coil that induces the magnetic field.

As an alternative to overcome the above-described limitations, application of FUS has been proposed. FUS, when administered in frequency less than 700 KHz (less the typical frequency used in diagnostic ultrasound imaging), can create a highly focused region of ultrasound energy to be able to reach deep regions of a tissue that are not reachable by the TMS technique. In addition, the size of the FUS focus is in the order of few millimeters, therefore smaller regions of the tissue can be treated.

Followed by early evidence by several investigations (see Bachtold et al., Ultrasound Med Biol. 1998 May; 24(4): pp. 557-65; Rinaldi et al., Brain Res. 1991 Aug. 30; 558(1): pp. 36-42), which investigated the in vivo and in vitro utility of FUS for the modification of excitability of brain tissues and nerves, several methods for focusing and tracking ultrasound beam through the skull have been proposed as disclosed in US20040236253, in which multi-arrayed actuation of small ultrasound transducers around the target area is used to create a steerable means of sonication to regional brain. This method, however, requires the estimation of the skull geometry using X-ray computed tomography (CT) to control the actuation timing (thus phase) of the transducer operation with the heat ablative procedures.

Bystritsky et al. (US20070299370) offered the method of using FUS to change the neuronal current in a biological tissue. This method, however, requires direct guidance of MRI to modify the current flow in the tissue. In addition, this method is not applicable in case where patients are with metal implants and claustrophobia. Further, transcranial delivery of FUS can only be achieved by the estimation of the skull thickness from the CT data, and fixation of the head with respect to the sonication transducer array via skull screw, which involves significantly painful procedure (and hair shaving) and recovery time from the surgical procedure.

There is, however, still a need for a simpler apparatus or method for non-invasive delivery of FUS generated by an ultrasound transducer to a targeted area of a biological tissue and real-time tracking of the focus of the ultrasound transducer.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

In one aspect, the present invention provides an apparatus for non-invasive delivery of focused ultrasound to a targeted area of a biological tissue. The apparatus comprises an ultrasound wave generator, a resonance unit, an ultrasound transducer, an applicator, and an ultrasound-tissue coupling bag.

The ultrasound wave generator includes an electrical function generator for generating a continuous current waveform with a specific frequency and a variable voltage amplitude, a pulse modulator operably connected to the electrical function generator for modulating amplitude of waveform, and a power amplifier operably connected to the pulse modulator (1B) for amplifying the modulated waveform.

The resonance circuit tunes the amplified waveform to have a predetermined frequency. The ultrasound transducer generates a focused beam of the tuned waveform. The applicator supports the ultrasound transducer. The ultrasound-tissue coupling bag is detachably mounted to the ultrasound transducer and holds therein a fluid that has acoustic impedance similar to that of the biological tissue.

In another aspect, the present invention provides a method for non-invasive delivery of focused ultrasound to modify a biological activity of the brain of a subject. The method comprises the steps of positioning a ultrasound transducer on or near a portion of the head of a subject, generating a ultrasound waveform (continuous current waveform), modulating the amplitude of the waveform, amplifying the modulated waveform, tuning the amplified modulated waveform to have a predetermined frequency, focusing the modulated waveform on a predetermined focal point, and adjusting the strength of the focused waveform and the position of the focal point.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a schematic diagram illustrating an apparatus for non-invasive delivery of focused ultrasound in accordance with an embodiment of the present invention;

FIG. 2 illustrates an example of the modulation of the amplitude of a waveform generated by the apparatus of FIG. 1;

FIG. 3 illustrates an example of the adjustment of the focal depth of an ultrasound transducer of the apparatus of FIG. 1;

FIG. 4 is a perspective view showing a dissembled state of an apparatus in accordance with an embodiment of the present invention;

FIGS. 5 to 7 are cross-sectional views of the apparatus of FIG. 4;

FIG. 8 is a perspective view of the apparatus of FIG. 4 operably coupled to a moving stage;

FIG. 9 illustrates an example of intra-MRI guided application of the apparatus of FIG. 4 to modify a biological activity of a human brain;

FIG. 10 illustrates another example of application of the apparatus of FIG. 4 to modify a biological activity of a human brain;

FIG. 11 illustrates still another example of application of the apparatus of FIG. 4 to modify a biological activity of a human brain without using the Radiological images; and

FIG. 12 is a flow chart showing a method for modifying a biological activity of a human brain.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

### DETAILED DESCRIPTION OF THE DISCLOSURE

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises” and/or “comprising” and the terms equivalent thereto, when used in this specification, specify the presence of stated features, integers, steps, operations, elements, and/or components, but do not preclude the presence or addition of one or more other features, integers, steps, operations, elements, components, and/or groups thereof.

As used herein, two or more components are “operably coupled” or “operably connected” when there are one or more connections between the components that allow or facilitate their functional interaction. For example, an electrical function generator would be “operably coupled” or “operably connected” to a pulse modulator when there is a functional attachment that allows the amplitude of a waveform generated by the electrical function generator to be used by the pulse modulator to modulate the amplitude of the waveform.

The excitability of a biological tissue can be modified by applying low-intensity (less than 3 Watt/cm² spatial-peak-time-averaged) ultrasound or high-intensity ultrasound. These modification has been achieved by way of, e.g., cavitation (in many cases, cavitation occurs in pressure greater than 2-3 mega Pascal) or heat-generation of local neural tissue.

In case where high-intensity ultrasound is used, heat is generated from the target area by the absorption of the acoustic energy, and the generated heat increases the tissue temperature 5-6° C. above the body temperature, thereby modifying a biological activity of the tissue. On the other hand, cavitation generated from the target area creates shockwaves to the tissue, thereby modifying a biological activity of the tissue.

According to the present invention, pulsed application of low-intensity FUS is used to induce mechanical agitation in a target area of an animal brain including a human brain without generation of such cavitation or heat, with an aim of inducing changes in neural cell excitability.

This mechanical agitation is translated to compressive or longitudinal waves to excitable biological tissues. The range of compressive motion is on the order of few nanometers whereas the longitudinal motion affects the shape of the cells in the tissues, on the order of few tens of micrometers. These motions, while not affecting the cell viability; are enough to modulate the activities of synaptic vesicles and ion channels of the cell membranes in the excitable cells. Accordingly, these motions result in temporary modification of the excitability of the tissue.

In one aspect, as discussed above, the present invention provides an apparatus for non-invasive delivery of FUS.

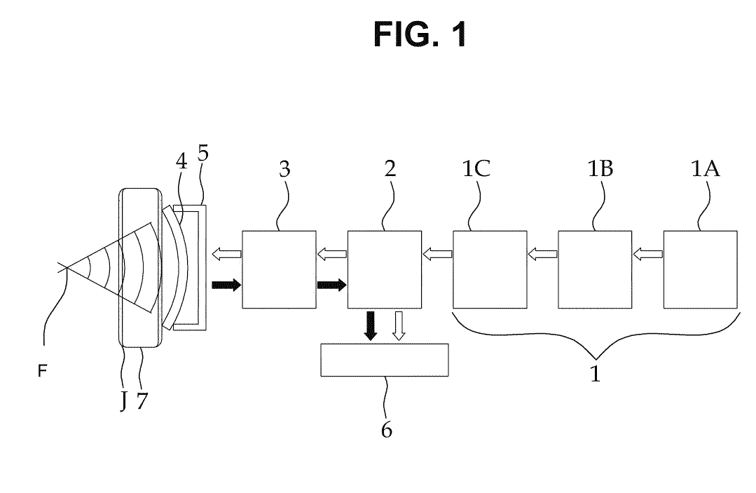

FIG. 1 is a schematic diagram illustrating an apparatus for non-invasive delivery of FUS to a targeted area of a biological tissue in accordance with an embodiment of the present invention. The apparatus comprises an ultrasound wave generator (1), a resonance circuit (3), an applicator (5), an ultrasound transducer (4), and an ultrasound-tissue coupling bag (7).

The ultrasound wave generator (1) includes an electrical function generator (1A), a pulse modulator (1B) operably connected to the electrical function generator (1A), and a power amplifier (10) operably connected to the pulse modulator (1B).

The electrical function generator (1A) functions to generate a continuous current waveform with a specific frequency and a variable voltage amplitude.

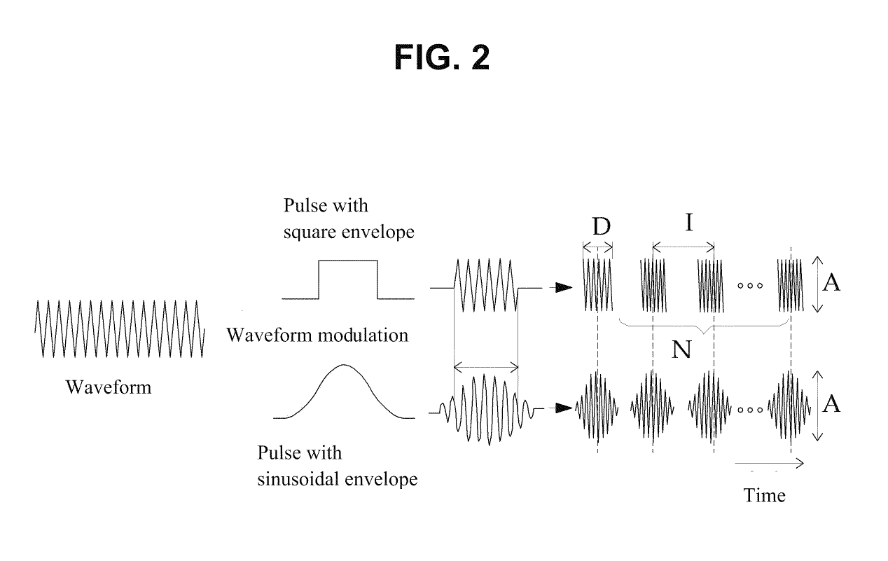

The pulse modulator (1B) functions to modulate the amplitude of the waveform generated by the electrical function generator (1A) to have a train of pulses with, but not limited to, square or sinusoidal envelope. The amplitude modulation can be performed by changing several parameters, as shown in FIG. 2. The parameters include a pulse duration (D), an inter-pulse-interval (I), an acoustic intensity (A), and the number of pulses (N). In detail, the acoustic intensity (A) can be changed by adjusting the amplitude of the electrical waveform generated by the function generator (1A). The pulse duration (D) can be modified by changing the number of wave cycles in the specific sonication frequency. The inter-pulse-interval (I) can be modified by adjusting the duty cycle of the function generator output. The number of the pulses (N) may determine the overall sonication duration and be controlled by adjusting the output duration of the electrical function generator (1A).

The electrical signal of the modulated waveform is not sufficient to drive the ultrasound transducer (4). Accordingly, the modulated waveform is amplified by the power amplifier (10). Preferably, a linear power amplifier may be used as the power amplifier (10) and the modulated waveform may be amplified to the range of 40 dB.

The resonance circuit (3) functions to tune the amplified signal to a predetermined frequency to maximize the power transfer efficiency.

The ultrasound transducer (4) functions to generate a focused beam of the tuned ultrasound and is supported by the applicator (5). The ultrasound transducer (4) has a predetermined curvature by which the focus (F) thereof is defined. Preferably, two or more ultrasound transducers (4) that can be detachably received in the applicator (5) and have different curvatures may be used, in which case focal depth of the sonication can be adjusted by selecting an appropriate transducer.

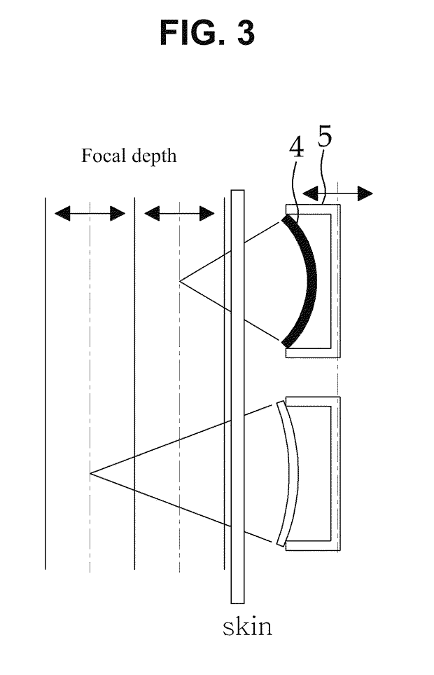

FIG. 3 illustrates an example of the adjustment of the focal depth. The upper ultrasound transducer (4) in the figure has a shorter focal depth (e.g. 7 cm) and the lower ultrasound transducer (4) has a longer focal depth (e.g. 9 cm). In addition, the respective ultrasound transducers can move toward and from a target surface by a predetermined length (e.g., 2 cm). The skin prohibits the ultrasound transducer (4) with a shorter focal depth from applying the focus deeper. In order to sonicate a target that is located deeper in the biological tissue, ultrasound transducers having different (i.e. greater radius-of-curvature (ROC); e.g. 9 cm) curvatures are used, and forward and backward movement of the transducer(s) itself enables a user of the apparatus to adjust the focal depth beyond the depth covered by the ultrasound transducer with a shorter focal depth. For example, two transducers, each having ROC of 7 cm and 9 cm respectively, can cover 4 cm in the target depth.

The ultrasound transducer (4) can be made of any material that allows the above-described function. Preferably, for example, the ultrasound transducer (4) may be made of a ceramic piezoelectric material.

The applicator (5) has a cylindrical shape with the top side open. The ultrasound transducer (4) is received in the applicator (5), preferably, along or near the circumference of the top side.

The ultrasound-tissue coupling bag (7) is detachably mounted to the ultrasound transducer (4). The ultrasound-tissue coupling bag (7) holds therein a fluid that has acoustic impedance similar to the biological tissue. A non-limiting example of the fluid is degassed water. The ultrasound-tissue coupling bag (7) may be made of, e.g., non-rubber, thin plastic or polymer materials with low acoustic absorption.

The apparatus, in certain embodiments, may further include a directional RF coupler (2) between the ultrasound wave generator (1) and the resonance circuit (3). The directional RF coupler (2) is coupled to a power sensor (6). The directional RF coupler (2) coupled to the power sensor (6) measures the electrical power supplied to the ultrasound transducer (4) and the electrical power reflected from the resonance circuit (3). The difference between the two electrical powers can be used to calculate the ultrasonic energy actually delivered to a target. The ultrasound transducer (4) itself can be calibrated to relate the amplitude of electrical signal input to the acoustic power output. It can be achieved by measuring the range of acoustic pressures through the use of calibrated needle hydrophone or membrane hydrophone.

In application of the apparatus to modify a biological activity of a subject, the focus of the ultrasound transducer (4) (sonication focus, F) may be tracked to provide image guidance. To enable the tracking, the apparatus may further include one or more markers that can be detected and imaged by appropriate detector(s). The number of the markers can be appropriately selected as long as the tracking can be performed. Preferably, at least two MRI-CT markers positioned so as to form an imaginary straight line with the sonication focus (F) of the ultrasound transducer (4) can be used. Also preferably, at least three IR markers having predetermined spatial relationship with the sonication focus (F) can be used. For example, the IR markers can be positioned on an imaginary plane and the center of the IR markers and the sonication focus (F) are on an imaginary straight line forming a predetermined angle with the plane. Preferably, the center of the IR markers and the sonication focus (F) are on an imaginary straight line perpendicular to the plane.

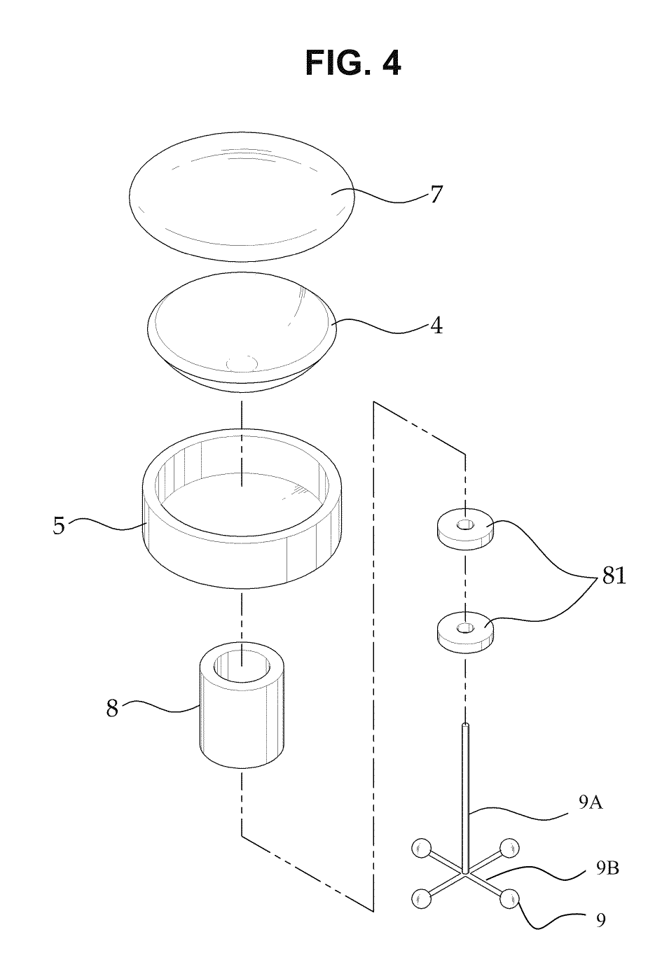

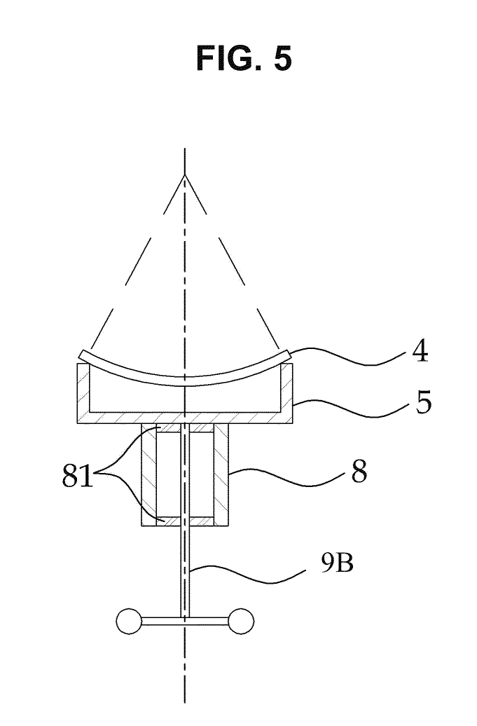

In an embodiment, for example, as shown in FIGS. 4 and 5, the apparatus may include two MRI-CT markers (81) and four infrared reflection (IR) makers (9).

The two MRI-CT markers (81) are fixed to a predetermined portion of the inner surface of a maker holder (8) attached to a predetermined portion of the bottom of the applicator (5) and are spaced apart from each other by a predetermined distance(s). The two MRI-CT markers (81) are spaced from the sonication focus (F) such that the markers and sonication focus are formed on an imaginary straight line, thereby enabling the detection and tracking of the coordinates among the markers and the sonication focus.

The four IR markers (9) are attached to the respective ends of a cross-shaped rod (9A). A vertical rod (9B) perpendicularly extends from the center of the cross-shaped rod (9A). The vertical rod (9B) passes through the holes of the MRI-CT markers fixed in the inner surface of the marker holder (8). With the IR markers, the sonication focus (F) with predetermined spatial relations to the IR markers can be detected by stereoscopic camera(s) with a tracking algorithm known in the art. In an embodiment, three IR markets (9) can be used.

Although the apparatus according to the embodiment shown in the drawings includes both the MRI-CT markers and the IR markers, an apparatus according to another embodiment can include the MRI-CT markers only or IR markers only.

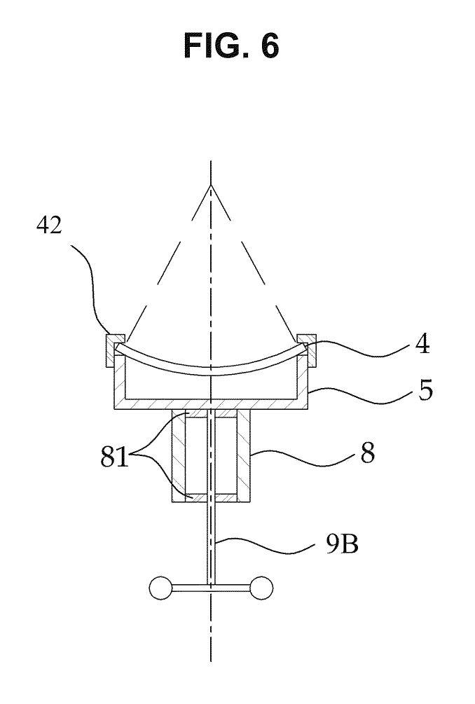

As discussed above, the ultrasound transducer (4) can be detached from the applicator (5). FIG. 6 shows an example of the detachable ultrasound transducer (4). As shown in FIG. 6, the ultrasound transducer (4) can be attached and detached by using a clip (42) or any other means to provide such function (e.g., a hook, a Velcro, etc.).

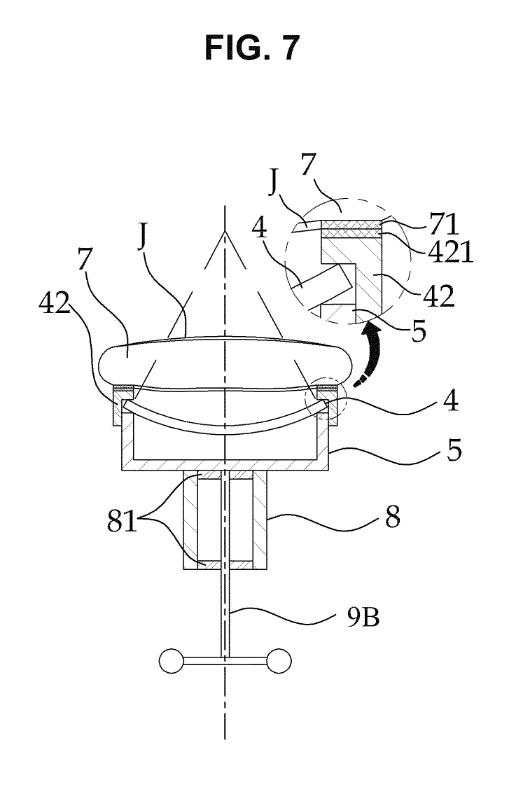

Likewise, in an embodiment, the ultrasound-tissue coupling bag (7) can be detached from the ultrasound transducer (4). FIG. 7 shows an example thereof. As shown in FIG. 7, the ultrasound-tissue coupling bag (7) can be attached and detached by an adhesive tape (421, 71) or any other means to provide such function. The ultrasound-tissue coupling bag (7) should be wide enough to transmit the entire acoustic field to a target and should provide air-tight contact between the ultrasound transducer (4) and the skin of a subject above a targeted site. Hydrogel (J) may be applied to at least an outer portion of the ultrasound-tissue coupling bag (7) to provide uninterrupted path to the target.

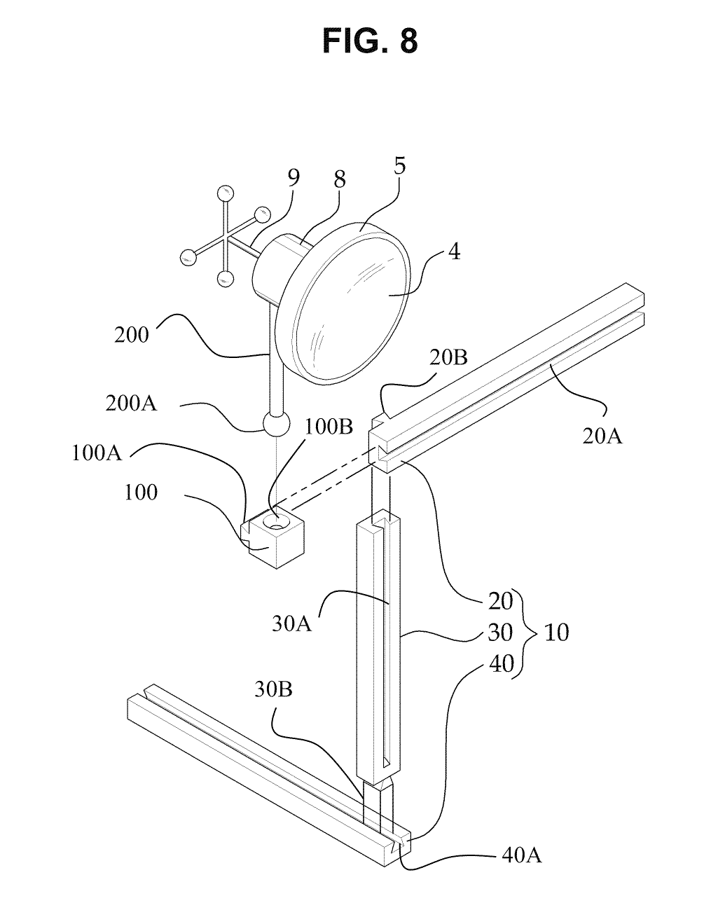

The apparatus according to the present invention may include a moving stage that allows the location of the sonication focus of the ultrasound transducer (4) to be changed. FIG. 8 shows an example thereof. The moving stage (10) of FIG. 8 includes an X-axis guide (20), a Y-axis guide (30), a Z-axis guide (40), and a rotational joint (100). The X-axis guide (20) has a groove (20A) and a protruding portion (20B). The Y-axis guide (30) has a groove (30A) and a protruding portion (30B) at one end thereof. The Z-axis guide (40) has a groove (40A). The rotational joint (100) has a protruding portion (100A) and a recess (100B). The protruding portion (100A) of the rotational joint (100) can move along the groove (20A) of the X-axis guide (20). The protruding portion (20B) of the X-axis guide (20) can move along the groove (30A) of the Y-axis guide (30). The protruding portion (30B) of the Y-axis guide (30) can move along the groove (40A) of the Z-axis guide (40). The applicator (5) is connected to a connecting rod (200) having a spherical end (200A). The spherical end (200A) is received in the recess (100B) of the rotational joint (100).

Although it is not described in the drawings, at least one external motor can be provided to activate the movement of the guides and the motor(s) can be controlled, being coupled with, e.g., an MRI/CT. Accordingly, the sonication focus of the ultrasound transducer (4) can be manually or automatically changed.

The moving stage of FIG. 8, however, is a non-limiting example. One example of the alternatives is an angular stage with lockable joints that can provide sufficient degree of freedom in the change of the location of the sonication focus of the ultrasound transducer (4). In an embodiment, a commercially-available articulated stage/arm with more than 6 degrees of freedom (i.e. 3 translational and 3 rotational movement) can be used to move the applicator (5) housing the ultrasound transducer (4).

As discussed above, in application of the apparatus to modify a biological activity of a subject, the focus (F) of the ultrasound transducer (4) may be tracked by using one or more markers.

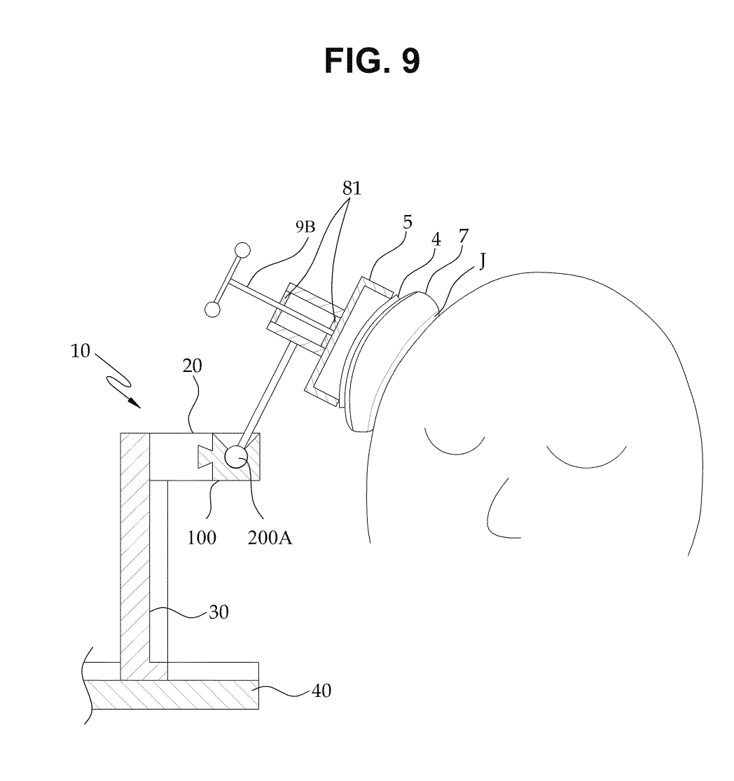

FIG. 9 illustrates an example of the application of the apparatus of FIG. 4 to modify a biological activity of a human brain. In this example, the apparatus is installed into an MRI magnetic bore such that the location of the sonication focus of the ultrasound transducer (4) can be detected and imaged by the MRI-CT marker (81) and a detector provided in the MRI/CT. The location of the focus can also be visualized and shown to the operator via the MRI/CT by imaging the whole setup with the person inside the MRI. Here, the location of the sonication focus can be imaged by any known imaging method such as MR acoustic radiation force imaging (ARFI). Although FIG. 9 shows that one moving stage is placed next to the subject human, additional moving stage(s) with independently controlled ultrasound transducer can also be placed to next to the subject human at the other location(s).

The human skull, with variable thickness approximately between 5-7 mm, can absorb, scatter, and reflect the ultrasound waves significantly. Therefore, the sonication will be administered through a thin (less than 2 mm) part of the temporal skull bone located behind and above each ear, as a ‘sonication window’. This part of the temporal bone, with the diameter approximately 2-3 cm, transmits the ultrasound with minimal distortion and absorption compared to the other parts of the skull.

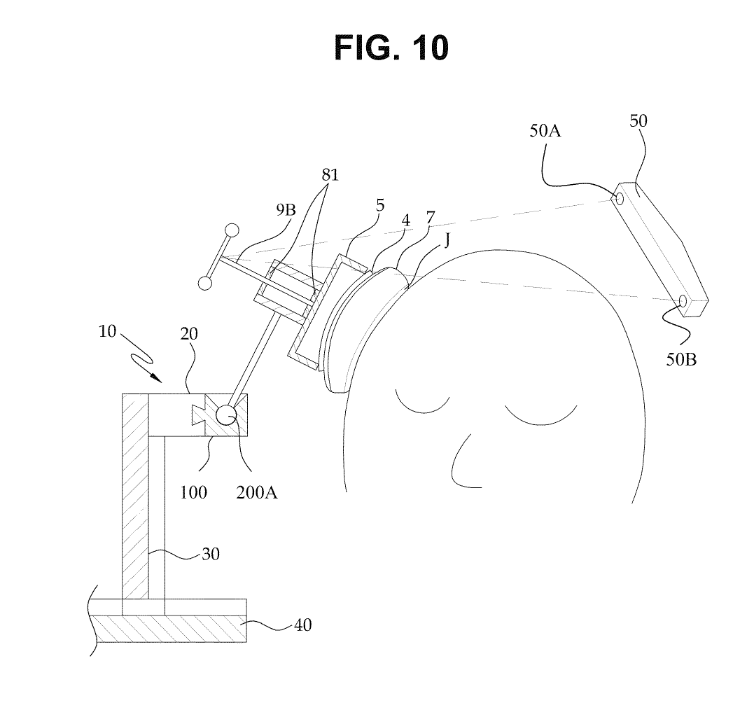

FIG. 10 illustrates another example of the application of the apparatus of FIG. 4 to modify a biological activity of a human brain. In this example, the apparatus is used outside MRI field, for example, in a doctor's office. In this case, an IR source/detector (50) including multiple (stereoscopic) cameras (50A, 50B) is provided. As the operator changes the location of the sonication focus, the IR markers (9) are imaged by the multiple (stereoscopic) cameras and reflected marker geometry can be used to detect the spatial coordinate of the sonication focus.

The tracking of the sonication focus can also be made by a plurality of laser beam generators. The number of the laser beam generators can be appropriately selected as long as the tracking can be performed. Preferably, at least two laser beam generators can be used.

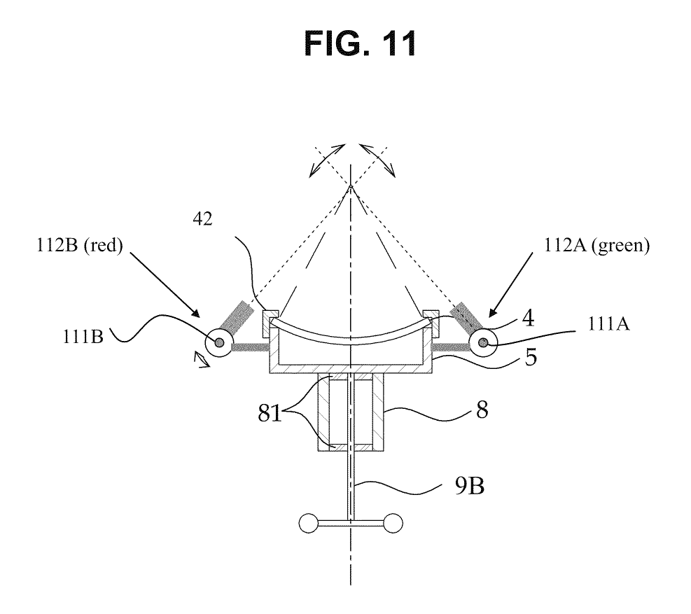

FIG. 11 illustrates an example of the application of the apparatus of FIG. 4 to modify a biological activity of a human brain. In this example, the apparatus includes two laser beam generators (111A, 111B) and two laser beam generators each being connected to the corresponding pivoting adapter (112A, 112B). Preferably, the laser beam generators (111A, 111B) can independently, pivotably move with respect to the applicator (5) by the pivoting adapters (112A, 112B) such that the two small laser beam sources with different colors (each having less than 1-2 mm in diameter), spaced opposite to each other with respect to the center of the sonication beam path, can be set to aim at the center of the acoustic focus, depending on the curvature of the ultrasound transducer (4).

Once the laser beams are set to the target, the surface over the sonication focus will be illuminated during the adjustment of the applicator. The beam path will be intersected by the surface and the spatial geometry of the spots illuminated by the lasers will be used to determine their intersecting points (i.e. sonication spot) in space. For example, the skin surface perpendicularly above the sonication target can be aimed using the 2 lasers aiming at the same location simultaneously. Then, the ultrasound transducer (4) can be moved toward to the focus while the spaces between laser spots to a predetermined level according to the geometrical relationship among the laser beams are monitored. In order to increase the accuracy of estimation, more than 2 laser beams can be used.

Although it is described in FIG. 11 that the apparatus includes the MRI-CT markers (81), the IR markers (9), and the laser beam generators (111A, 111B), an apparatus according to another embodiment can include the laser beam generators (111A, 111B) only.

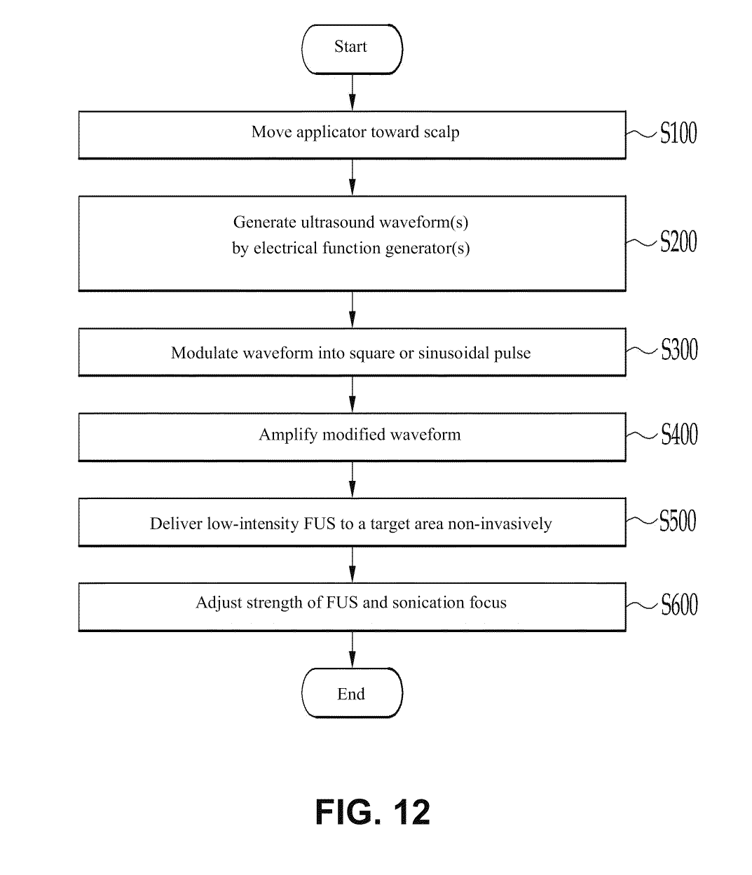

In another aspect, the present invention provides a method for non-invasive delivery of FUS to modify a biological activity of an animal brain using the above-described apparatus(es). FIG. 12 is a flow chart showing an example of the method with reference to a human brain.

In step S100, the applicator (5) is adjusted such that the ultrasound transducer (4) attached to the applicator (5) may be positioned on or near a portion of the head of a subject. As discussed above, in this step, the ultrasound-tissue coupling bag (7) containing degassed water may be detachably mounted to the ultrasound transducer (4) and a hydrogel (J) is applied on the both sides of the bag (7), which contributes FUS to be delivered to a target more accurately.

In step S200, at least one ultrasound waveform (continuous current waveform) is generated by the electrical function generator (1A).

In step S300, the amplitude of the waveform generated by the electrical function generator (1A) is modulated by the pulse modulator (1B) to have a train of pulses with, but not limited to, square or sinusoidal envelope.

In step S400, the modulated waveform is amplified by the power amplifier (10).

In step S500, the modulated waveform is tuned by the resonance circuit (3) and focused by the ultrasound transducer (4). Preferably, the amplitude-modulated electrical signals may be tuned for the range of 200 kHz-700 Khz, which allows the ultrasound delivery through the skull (part of the temporal bone) without significant distortion of the ultrasound field.

In step S600, the strength of the focused ultrasound and the position of the ultrasound focus are adjusted. For example, in order to induce excitation of the excitable tissue, the acoustic intensity of the ultrasound may be adjusted to be between 1-65 Watt/cm² (Spatial peak pulse averaged), at a rate of 1 to 2000 Hz with the pulse duration of 1-100 msec, with total sonication duration less than 2 sec. On the other hand, in order to suppress the excitable tissue, the acoustic intensity of the ultrasound may be adjusted to be between 1-50 Watt/cm² (Spatial peak pulse averaged), at a rate of 100 to 2000 Hz with the pulse duration of less than 1 msec, for the duration more than 2 sec.

In an embodiment, the method may further include a step of visualizing the ultrasound focus in an MRI or CT image in which the brain of a subject is shown. Visualization of the ultrasound focus can be done as follows. A person undergoing the sonication takes volumetric high-resolution (on the order of 1×1×1 mm³ voxel size) MRI or CT with at least three surface image markers on the forehead including the skin surface behind the ear lobes. The center point of these fiducial image markers is marked with a non-permanent ink to allow the operator to identify the fiducial marker location. After acquiring the volumetric information (virtual space), the real physical space of the person's anatomy is mathematically registered to the virtual space. With respect to the real space of the person's anatomy, the location of the sonication focus as well as the path of the sonication (as vector) are calculated and displayed to the virtual space using the mathematical algorithm (e.g., rigid 3D transformation matrix), thus providing the operator with the exact location of the sonication focus. CT imaging may be optionally necessary to locate the site of a thin temporal bone to be served as acoustic windows for the purpose the brain sonication.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. For example, although the present invention is described with the examples of a human brain, it can be applied to the brains of the other animals. It is intended that the scope of the invention be defined by the Claims appended hereto and their equivalents.

1. An apparatus for non-invasive delivery of focused ultrasound to a targeted area of a biological tissue, the apparatus comprising:
an ultrasound wave generator (1) which includes an electrical function generator (1A) for generating a continuous current waveform with a specific frequency and a variable voltage amplitude, a pulse modulator (1B) operably connected to the electrical function generator (1A) for modulating amplitude of waveform, and a power amplifier (10) operably connected to the pulse modulator (1B) for amplifying the modulated waveform;
a resonance circuit (3) for tuning the amplified waveform to have a predetermined frequency;
an ultrasound transducer (4) for generating a focused beam of the tuned waveform;
an applicator (5) supporting the ultrasound transducer (4); and
an ultrasound-tissue coupling bag (7) detachably mounted to the ultrasound transducer (4) and holding therein a fluid that has acoustic impedance similar to that of the biological tissue.

2. The apparatus of claim 1, wherein the applicator (5) has a cylindrical shape with the top side open and the ultrasound transducer (4) is received in the open top of the applicator (5).

3. The apparatus of claim 1, further comprising a directional RF coupler (2) between the ultrasound wave generator (1) and the resonance circuit (3), wherein the directional FR coupler (2) is coupled to a power sensor (6) for measuring the electrical power supplied to the ultrasound transducer (4) and that reflected from the resonance circuit (3).

4. The apparatus of claim 1, further comprising at least two markers that allows the focus of the ultrasound transducer (4) to be detected and tracked in space.

5. The apparatus of claim 4, further comprising at least two MRI-CT markers (81), wherein the two MRI-CT markers (81) are spaced from each other by a predetermined distance and are spaced from the focus of the ultrasound transducer (4) by a predetermined distance such that the MRI-CT markers (81) and the focus are defined on an imaginary straight line.

6. The apparatus of claim 4, further comprising at least three infrared reflection (IR) markers (9), wherein the IR markers are positioned on an imaginary plane and the center of the IR markers and the sonication focus (F) are on an imaginary straight line forming a predetermined angle with the plane.

7. The apparatus of claim 6, wherein the predetermined angle is 90° or substantially 90°.

8. The apparatus of claim 6, further comprising at least two MRI-CT markers (81), wherein the two MRI-CT markers (81) are spaced from each other by a predetermined distance and are spaced from the focus of the ultrasound transducer (4) by a predetermined distance such that the MRI-CT markers (81) and the focus are defined on an imaginary straight line.

9. The apparatus of claim 1, further comprising at least two laser beam generators (111A, 111B), each of which is connected to a corresponding pivoting adapter (112A, 112B) that is attached to the applicator (5) for allowing the laser beam generators to be pivotably moved.

10. The apparatus of claim 9, further comprising further comprising at least two MRI-CT markers (81), wherein the two MRI-CT markers (81) are spaced from each other by a predetermined distance and are spaced from the focus of the ultrasound transducer (4) by a predetermined distance such that the MRI-CT markers (81) and the focus are defined on an imaginary straight line.

11. The apparatus of claim 9, further comprising at least three infrared reflection (IR) markers (9), wherein the IR markers are positioned on an imaginary plane and the center of the IR markers and the sonication focus (F) are on an imaginary straight line forming a predetermined angle with the plane.

12. The apparatus of claim 11, further comprising further comprising at least two MRI-CT markers (81), wherein the two MRI-CT markers (81) are spaced from each other by a predetermined distance and are spaced from the focus of the ultrasound transducer (4) by a predetermined distance such that the MRI-CT markers (81) and the focus are defined on an imaginary straight line.

13. The apparatus of claim 1, wherein the applicator is mounted to a moving stage.

14. A method for non-invasive delivery of focused ultrasound to modify a biological activity of the brain of a subject, the method comprising the steps of:
positioning a ultrasound transducer on or near a portion of the head of a subject;
generating a ultrasound waveform (continuous current waveform);
modulating the amplitude of the waveform;
amplifying the modulated waveform;
tuning the amplified modulated waveform to have a predetermined frequency;
focusing the modulated waveform on a predetermined focal point; and
adjusting the strength of the focused waveform and the position of the focal point.

15. The method of clam 14, further comprising the step of detachably mounting an ultrasound-tissue coupling bag containing degassed water and applying a hydrogel on the both sides of the bag.

16. The method of clam 14, wherein in the step of tuning, the amplified modulated waveform is tuned for the range of 200 kHz-700 Khz.

17. The method of claim 14, wherein in the step of adjusting, the strength is adjusted to be between 1-65 Watt/cm² (Spatial peak pulse averaged), at a rate of 1 to 2000 Hz with the pulse duration of 1-100 msec, with total sonication duration less than 2 sec.

18. The method of claim 14, wherein in the step of adjusting, the strength is adjusted to be between 1-50 Watt/cm² (Spatial peak pulse averaged), at a rate of 100 to 2000 Hz with the pulse duration of less than 1 msec, for the duration more than 2 sec.

19. The method of claim 14, further comprising a step of visualizing the focus in an MRI or CT image in which the brain of a subject is shown.
